(19) United States
(12) Patent Application Publication
(10) Pub. No.: US 2026/0331292 A1
(43) Pub. Date: Oct. 22, 2026

(54) A BLOOD GENE SCORE TO PROGNOSE CHRONIC LUNG ALLOGRAFT DYSFUNCTION

(71) Applicants: INSTITUT NATIONAL DE LA SANTÉ ET DE LA RECHERCHE MÉDICALE, Paris (FR); NANTES UNIVERSITÉ, Nantes (FR); CENTRE HOSPITALIER UNIVERSITAIRE DE NANTES, Nantes (FR); CENTRE NATIONAL DE LA RECHERCHE SCIENTIFIQUE, Paris (FR)

(72) Inventors: Richard Danger, Nantes (FR); Sophie Brouard, Paris (FR); Adrien Tissot, Nantes (FR); Antoine Magnan, Nantes (FR); Mallory Pain, Paris (FR)

(21) Appl. No.: 19/486,302

(22) PCT Filed: May 31, 2024
(86) PCT No.: PCT/EP2024/065111

§ 371 Date: Nov. 20, 2025

(30) Foreign Application Priority Data
Jun. 2, 2023 (EP) 23305873.4

(51) Int. Cl. G16H 50/30; G01N 33/68

(52) U.S. Cl. CPC G16H 50/30; G01N 33/6884; G01N 33/6893

(57) ABSTRACT

The present invention relates to a method for assessing the risk of Chronic Lung Allograft Dysfunction (CLAD) in a lung transplanted subject, comprising determining a risk score based on biological and clinical parameters. The present invention further relates to a method for preventing CLAD occurrence in a subject, comprising determining the personalized course of treatment for the subject based on the obtained risk score.

### FIELD OF INVENTION

The present invention relates to the field of allograft dysfunction. In particular, the invention relates to a method for assessing the risk of Chronic Lung Allograft Dysfunction (CLAD) in a lung transplanted subject. The present invention further relates to determine a risk score based on biological and clinical parameters.

### BACKGROUND OF INVENTION

Chronic lung allograft dysfunction (CLAD) is an irreversible and progressive form of allograft failure in lung transplantation and represents the major limitation to long-term survival after lung transplantation. It occurs in ~50% of lung transplanted patients at 5 years post-transplantation and results to retransplantation or death of the recipient.

Two main phenotypes of CLAD have been defined: the bronchiolitis obliterans syndrome (BOS) and the restrictive allograft syndrome (RAS). BOS is considered as the most common manifestation of CLAD (with 35% at 5 years), characterized by obstruction of the small airways by tissue remodeling and extracellular matrix deposition with obstructive respiratory functional tests. RAS is characterized by a major fibrotic process and functional restrictive syndrome.

In clinic, CLAD is discovered lately and only based on a decrease of pulmonary function measured by the forced expiratory volume in 1 second (FEV1). Briefly, ISHLT guidelines states that CLAD is suspected by a decrease of 10% of respiratory function and diagnosed based on a sustained decline in FEV1 by at least 20% from the subject's reference (baseline) value.

Currently, no treatment is available to reverse CLAD after diagnosis. Identify early biomarkers of CLAD or prognostic method is therefore a major challenge and will allow to establish new therapeutic strategies to reverse the progression of the disease.

Several biomarkers have been proposed as associated with CLAD diagnosis (such as cell-free DNA in plasma, cell count in bronchoalveolar lavages . . . ), but none are currently implemented in routine.

Accordingly, there is a need to determine a prognostic method, especially by identifying specific markers or parameters for which combination could help to identify patients at risk of CLAD, so as to prevent lung allograft damage.

### SUMMARY

The present invention relates an in vitro non-invasive prognostic method for assessing a risk of Chronic Lung Allograft Dysfunction (CLAD) in a subject, comprising:
a. obtaining at least one variable from the subject by measuring the level, amount or concentration of at least one biomarker in a biological sample from the subject, wherein said at least one biomarker is selected from the group comprising TCL1A, BLK, POU2AF1 and matrix metalloproteinase-9 (MMP-9),
b. obtaining at least one clinical data variable from the subject selected from the group comprising allograft rejection experience before sampling, maintenance treatment at sampling, induction treatment at transplantation, and mathematical combinations thereof,
c. obtaining a risk score by mathematically combining in a multivariate model:
   i. said at least one variable obtained in step (a) and/or a reduction dimension of the level, amount or concentration mathematically obtained from at least two biomarkers measured in step (a); and
   ii. said at least one clinical data variable obtained in step (b), and
d. determining the risk of Chronic Lung Allograft Dysfunction in the subject by comparing the obtained risk score with a reference risk score.

In one embodiment, the in vitro non-invasive prognostic method as described above, comprises obtaining a risk score by mathematically combining in a multivariate model in step (c) at least the following variables:
i) the level, amount or concentration of TCL1A, BLK, and POU2AF1, and the clinical data variable allograft rejection experience before sampling, or
ii) the level, amount or concentration of TCL1A, BLK, and POU2AF1, and the clinical data variable maintenance treatment at sampling, or
iii) the level, amount or concentration of TCL1A, BLK, and POU2AF1, and the clinical data variable induction treatment at transplantation.

The present invention also relates to an in vitro non-invasive prognostic method for assessing the risk of Chronic Lung Allograft Dysfunction (CLAD) in a subject, comprising:
a. obtaining at least one variable from the subject by measuring the expression level of at least one biomarker in a biological sample from the subject, wherein said at least one biomarker is selected from TCL1A, BLK, POU2AF1 and matrix metalloproteinase-9 (MMP-9),
   a1. optionally mathematically obtaining a reduction dimension of expression of at least two of the biomarkers measured in step (a),
b. obtaining at least one clinical data variable selected from the group comprising allograft rejection experience before sampling, maintenance treatment at sampling, and induction treatment at transplantation, and mathematical combinations thereof, and
c. mathematically combining in a multivariate model
   i. said at least one variable obtained in step (a) and/or said reduction dimension of expression of the at least two biomarkers when obtained in step (a1); and
   ii. said at least one clinical data variable obtained in step (b);
thereby obtaining a risk score.

In one embodiment, the in vitro non-invasive prognostic method, comprises obtaining a risk score by mathematically combining in a multivariate model in step (c) at least the following variables:
i) the level, amount or concentration of TCL1A, BLK and POU2AF1; and the clinical data variable allograft rejection experience before sampling, or
ii) the level, amount or concentration of TCL1A, BLK and POU2AF1, and the clinical data variable maintenance treatment at sampling, or
iii) the level, amount or concentration of TCL1A, BLK and POU2AF1, and the clinical data variable induction treatment at transplantation.

In one embodiment, the subject is a lung transplanted subject.

In one embodiment, the risk score is suitable to predict the risk of having a bronchiolitis obliterans syndrome (BOS).

In one embodiment, the risk score is suitable to predict the risk of having a restrictive allograft syndrome (RAS).

In one embodiment, the level, amount or concentration of the at least one biomarker is measured in a biological sample from 18 months post-transplantation, preferably 24 months post-transplantation.

In one embodiment, the biological sample is a blood sample.

In one embodiment, the multivariate model is obtained from a Cox model, and/or wherein said multivariate model is time-fixed or time-dependent, preferably is time-dependent.

In one embodiment, the in vitro non-invasive prognostic method is computer implemented.

The present invention further relates to a method for preventing the risk of having CLAD in a subject, comprising
a. a first step consisting in implementing the in vitro non-invasive prognostic method according to the invention as described hereinabove, so as to obtain a risk score,
b. determining a risk of having CLAD for the subject based on the obtained risk score, and
c. determining the personalized course of treatment for the subject based on the obtained risk score.

In one embodiment, when it is concluded at step b) that the subject is at risk of having CLAD, the subject is thus eligible to preventive or therapeutic treatment.

In one embodiment, when it is concluded at step b) that the subject is not at risk of having CLAD, the subject is thus eligible to for immunosuppressive therapy minimization and lower frequency of clinical follow-up.

In one embodiment, the preventive or therapeutic treatment is selected from the group comprising: immunosuppressive and immunomodulatory therapies drugs, anti-fibrotic treatment, cellular-based therapies, such as the use of mesenchymal stromal cells, endothelial progenitor cells, regulatory T cells (Tregs) or chimeric antigen receptors (CAR) Tregs, prophylactic treatment with azithromycin, total lymphoid irradiation (TLI) or extracorporeal photopheresis (ECP), current ant novel biotherapy such as anti-TNFα, anti-IL6R or JAKs inhibitors.

In one embodiment, the subject is susceptible to have BOS or the subject is susceptible to have RAS.

### DEFINITIONS

In the present invention, the following terms have the following meanings:

“Allograft transplant” refers to cells, tissues or organs transplantation, wherein the recipient and the donor are of the same species but are genetically non-identical.

“Biomarker” refers to a characteristic that is objectively measured and evaluated as an indicator of normal biologic processes, pathogenic processes, or pharmacologic responses to a therapeutic intervention. A biomarker may be used to diagnose a specific infection and/or disease as early as possible (diagnostic biomarker), to predict the risk of developing an infection and/or a disease (risk biomarker), to predict the evolution of an infection and/or a disease (prognostic biomarker), to predict the response and the toxicity to a given treatment (companion biomarker).

“BOS” refers to bronchiolitis obliterans syndrome. BOS is the main CLAD subtype. BOS is characterized by bronchiolar inflammation with fibrosis of resulting in progressive and irreversible airflow obstruction.

“Biological sample” refers to any sample obtained from a subject, preferably a transplanted subject, such as a blood sample (including whole blood, a serum sample, a plasma sample, blood cells), a bronchoalveolar lavage or a tissue biopsy.

“BLK” refers to B-cell lymphocyte kinase, a non-receptor tyrosine kinase. BLK has a role in B-lymphocyte development, differentiation and signaling. The naturally occurring human BLK gene has a nucleotide sequence as shown in Genbank Accession number NM_001715.2 and the naturally occurring human BLK protein has an aminoacid sequence as shown in Genbank Accession number NP_001706.2. The murine nucleotide and amino acid sequences have also been described (Genbank Accession numbers NM_007549.2 and NP_031575.2).

“CLAD” refers to chronic lung allograft dysfunction. CLAD is an irreversible and progressive form of allograft failure in lung transplantation and represents the major limitation to long-term survival after lung transplantation. It occurs in ~50% of lung transplanted patients at 5 years post-transplantation and results to retransplantation or death of the recipient. CLAD has two major subtypes, bronchiolitis obliterans syndrome (BOS) and restrictive allograft syndrome (RAS).

“MMP-9” refers to Matrix metalloproteinase 9. In the context of the invention MMP-9 is a secretory endopeptidase which has been identified as key mediator in processes associated with CLAD. The naturally occurring human MMP-9 gene has a nucleotide sequence as shown in Genbank Accession number NM_004994.3 and the naturally occurring human MMP-9 protein has an aminoacid sequence as shown in Genbank Accession number NP_004985.2. The murine nucleotide and amino acid sequences have also been described (Genbank Accession numbers NM_013599.5 and NP_038627.1).

“POU2AF1” refers to refers to POU Class 2 Homeobox Associating Factor 1. The naturally occurring human POU2AF1 gene has a nucleotide sequence as shown in Genbank Accession number NM_006235 and the naturally occurring human POU2AF1 protein has an amino acid sequence as shown in Genbank Accession number NP_006226.2. The murine nucleotide and amino acid sequences have also been described (Genbank Accession numbers NM_011136.2 and NP_035266.1).

“RAS” refers to restrictive allograft syndrome. RAS is a CLAD subtype. RAS is characterized by peripheral lung fibrosis (i.e., in the visceral pleura, in the alveolar interstitium and in the interlobular septa), resulting in a reduction of total lung capacity.

“Score” refers to any digit value obtained by the mathematical combination (univariate or multivariate) of at least one biomarker and/or at least one clinical data variable and/or at least one physical data and/or at least one binary marker and/or at least one blood test result. In one embodiment, a score is an unbound digit value. In another embodiment, a score is a bound digit value, obtained by a mathematical function. A score may be a binary value (0 or 1), a probability value, a percentage value, an integer value, or a decimal value.

“Subject” refers to an animal, including a human. In the sense of the present invention, a subject may be a patient, i.e. a person receiving medical attention, undergoing or having underwent a medical treatment, or monitored for the development of a disease. In the context of the present invention, the subject is a transplanted subject, preferably a lung transplanted subject.

“TCL1A” refers to T-cell leukemia or lymphoma protein 1 A which is a protein that in humans encoded by the TCL1A gene. The naturally occurring human TCL1A gene has a nucleotide sequence as shown in Genbank Accession numbers NM_021966.2 (variant 1) and NM_001098725.1 (variant 2), and the naturally occurring human TCL1A protein has an amino acid sequence as shown in Genbank Accession numbers NP 068801.1 (variant 1) and NP 001092195.1 (variant 2). The naturally occurring murine TCL1A gene has a nucleotide sequence as shown in Genbank Accession numbers NM_009337.3 (variant 1), NM_001289468.1 (variant 2), NM_001309485.1 (variant 4) and NM_001309484.1 (variant 5) and the naturally occurring murine TCL1A protein has an amino acid sequence as shown in Genbank Accession numbers: NP_033363.1 (variant 1), NP_001276397.1 (variant 2), NP 001296414.1 (variant 4) and NP 001296413.1 (variant 5).

### DETAILED DESCRIPTION

The present invention relates to in vitro prognostic method, preferably to non-invasive in vitro prognostic methods, for prognosing Chronic Lung Allograft Dysfunction (CLAD) in a subject. Accordingly, the methods of the invention may also be defined as a non-invasive in vitro prognostic methods for assessing the risk of Chronic Lung Allograft Dysfunction (CLAD) in a subject. In other words, the method of the invention allows to assess the risk for a subject of developing/having a Chronic Lung Allograft Dysfunction (CLAD).

In what follows, the term “mathematically” may denote the use of calculations, computations, processing approaches, operations, or procedures based on mathematical principles and reasoning. For example, a data on which we apply an operation (e.g. normalization) is a mathematically obtained data.

This invention thus relates to an in vitro non-invasive prognostic method for assessing a risk of Chronic Lung Allograft Dysfunction (CLAD) in a subject, comprising:
a. obtaining at least one variable from the subject by measuring the level, amount or concentration of at least one biomarker in a biological sample from the subject, wherein said at least one biomarker is selected from the group comprising or consisting of TCL1A, BLK, POU2AF1 and matrix metalloproteinase-9 (MMP-9),
b. obtaining at least one clinical data variable from the subject selected from the group comprising or consisting of allograft rejection experience before sampling, maintenance treatment at sampling, induction treatment at transplantation, and mathematical combinations thereof, and
c. obtaining a risk score by mathematically combining in a multivariate model:
   i. said at least one variable obtained in step (a) and/or a reduction dimension of the level, amount or concentration mathematically obtained from at least two biomarkers measured in step (a); and
   ii. said at least one clinical data variable obtained in step (b); and
d. determining the risk of Chronic Lung Allograft Dysfunction in the subject by comparing the obtained risk score with a reference risk score.

This invention thus relates to an in vitro non-invasive prognostic method for assessing the risk of Chronic Lung Allograft Dysfunction (CLAD) in a subject, comprising:
a. obtaining at least one variable from the subject by measuring the expression level of at least one biomarker in a biological sample from the subject, wherein said at least one biomarker is selected from the group comprising or consisting of TCL1A, BLK, POU2AF1 and matrix metalloproteinase-9 (MMP-9),
   a1. optionally mathematically obtaining a reduction dimension of the expression (i.e. dimensionality reduction of expression) of at least two of the biomarkers measured in step (a),
b. obtaining at least one clinical data variable selected from the group comprising or consisting of allograft rejection experience before sampling, maintenance treatment at sampling, induction treatment at transplantation, and mathematical combinations thereof, and
c. mathematically combining in a multivariate model
   i. said at least one variable obtained in step (a) and/or said reduction dimension of expression of the at least two biomarkers when obtained in step (a1.); and
   ii. said at least one clinical data variable obtained in step (c);
      thereby obtaining a risk score.

Combining in a multivariate model may refer to using a multivariate model to join individual contribution of variables such as the at least one variable, the at least one clinical data variable obtained in step (b) and/or the dimension reduction of expression or of the level, amount or concentration mathematically obtained from at least two biomarkers measured in step (a). For example, the multivariate model may implement a linear regression, a logistic regression, or a machine learning algorithm like random forests, neural networks or support vector machines.

Advantageously, a multivariate model captures and analyzes the relationships between multiple variables, for instance the combined variables. By considering these relationships between variables, a multivariate model may offer accurate predictions compared to univariate or bivariate approaches.

Combining variables in a multivariate model may comprise:
collecting data on the combined variables of interest;
pre-processing the collected combined variables (e.g. data cleaning, handling missing values, formatting);
choosing a multivariate model based on the combined variables:
   for example, in case of multiple independent variables (e.g. biomarker expression levels or amount or concentration of biomarker and clinical data variables) and a continuous dependent variable (e.g., a risk score for Chronic Lung Allograft Dysfunction), a multivariate linear regression model may be suitable for modeling the relationship between these variables and predicting the risk score;
   in another example, if the outcome of interest is binary (e.g., whether a subject develops CLAD or not), and the multiple variables are independent variables, a multivariate logistic regression model may be appropriate to predict the probability of developing CLAD based on combined biomarker expression and clinical data,
fitting the multivariate model to the data on the combined variables using statistical techniques such as maximum likelihood estimation or least squares estimation, so as to obtain variables relationship parameters; for example, the obtained variables relationship parameters may be used to assess how well the multivariate model fits the data, to check for violations of assumptions, and/or to evaluate the accuracy of the risk score obtained by the multivariate model.
obtaining a risk score using the fitted multivariate model.

In one embodiment, the in vitro non-invasive prognostic method is for assessing the risk of having CLAD in a subject (or risk of CLAD in a subject, or risk of developing CLAD in a subject). In the context of the invention, the prognostic method is to determine into a group of subjects who will have CLAD or who will not have CLAD. In one embodiment, the term “assessing the risk” refers to assessing the probability according to which the subject will have CLAD. Such assessment is usually not intended by the skilled in the art to be correct for 100% of the subjects to be investigated.

In one embodiment, the subject is a mammal, preferably a human. In one embodiment, the subject is a male or a female. In one embodiment, the subject is an adult or a child.

In one embodiment, the subject is a transplanted subject. In one embodiment, the subject is a lung transplanted subject. In one embodiment, the subject is susceptible to have (or develop) CLAD. In one embodiment, the subject is susceptible to have (or develop) BOS. In one embodiment, the subject is susceptible to have (or develop) RAS. In one embodiment, the subject is susceptible to have (or develop) BOS and RAS.

In one embodiment, the in vitro non-invasive prognostic method comprises obtaining biological and clinical data variables. The biological data variable refers to the level, amount or concentration of biomarkers.

In one embodiment, the in vitro non-invasive prognostic method comprises obtaining biological and clinical data variables. In one embodiment, the biological data variable refers to the level, amount or concentration of biomarkers. In one embodiment, the level of biomarkers corresponds to the expression level of biomarkers. In one embodiment, the biological data variable refers to the expression level of biomarkers.

In one embodiment, the level, amount or concentration corresponds to the transcription level (i.e., the expression of mRNA) or to the translation level (i.e., the expression of the corresponding protein) of the at least one biomarker. In one embodiment, the level, amount or concentration of the at least one biomarker is determined at the RNA level, i.e., at the transcription level. Methods for determining the transcription level of a biomarker are well known in the art. Examples of such methods include, but are not limited to, real-time quantitative PCR (qPCR), RT-PCR, RT-qPCR, hybridization techniques (such as, e.g., using microarrays, NanoString® method, etc.), northern blot, and combination thereof, including, but not limited to, hybridization of amplicons obtained by RT-PCR, sequencing (such as, e.g., next-generation DNA sequencing or RNA-seq—also known as “whole transcriptome shotgun sequencing”), and the like.

In one embodiment, the level, amount or concentration of the at least one biomarker is determined at the protein level, i.e., at the translation level. Methods for determining the translation level of a biomarker are well known in the art. Examples of such methods include, but are not limited to, immunohistochemistry, multiplex methods (Luminex), western blot, enzyme-linked immunosorbent assay (ELISA), sandwich ELISA, flow cytometry, fluorescent-linked immunosorbent assay (FLISA), enzyme immunoassay (EIA), radioimmunoassay (RIA), mass spectrometry (such as, e.g., tandem mass spectrometry [MS/MS], chromatography-assisted mass spectrometry and combinations thereof), and the like.

In one embodiment, the level, amount or concentration can be expressed in terms of absolute or relative levels, amounts or concentrations.

When expressed in terms of relative levels, amounts or concentrations, the levels, amounts or concentrations are normalized relative to the level, amount or concentration of one or several reference markers. A “reference marker” may also be referred to as “housekeeping marker” and can be a “housekeeping gene” if the level, amount or concentration of the at least one biomarker is determined at the RNA level; or a “housekeeping protein” if the level, amount or concentration of the at least one biomarker is determined at the protein level. The term “housekeeping marker” hence refers to a gene or a protein, constitutively expressed and necessary for basic maintenance and essential cellular functions. A housekeeping marker is generally not expressed in a cell- or tissue-dependent manner, most often being expressed by all cells in a given organism. Housekeeping markers also have a relatively stable or steady expression; hence they serve as suitable markers to normalize levels, amounts or concentrations of biomarkers of interest. Housekeeping markers and their use in data normalization are well known in the art.

In one embodiment, the biomarkers TCL1A, BLK, POU2AF1 and/or MMP-9 are identified as predictive biomarkers of CLAD. In one embodiment, the predictive biomarkers are identified using a metric model. In one embodiment, the metric model used is the area under the curve and/or the precision-recall area under the curve (Precision re-call AUC). The higher is the AUC, the higher is the accuracy of prediction.

In one embodiment, the level, amount or concentration of at least one biomarker selected from the group comprising or consisting of TCL1A, BLK, POU2AF1 and MMP-9 is obtained from a biological sample. In one embodiment, the sampling of the biological samples is obtained at the time of transplantation and then every 6 months up to 5 years post transplantation.

In one embodiment, the level, amount or concentration of the at least one biomarker is measured in a biological sample at 0-, 6-, 12-, 18-, 24- and 30-months post-transplantation. In one embodiment, the level, amount or concentration of the at least one biomarker is measured in a biological sample from 18 months post-transplantation, preferably 24 months post-transplantation.

In one embodiment, the in vitro non-invasive prognostic method comprises a step of determining the level, amount or concentration of TCL1A in a sample, preferably the expression level of TCL1A in a sample.

In one embodiment, the in vitro non-invasive prognostic method comprises a step of determining the level, amount or concentration of BLK in a sample, preferably the expression level of BLK in a sample.

In one embodiment, the in vitro non-invasive prognostic method comprises a step of determining the level, amount or concentration of POU2AF1 in a sample, preferably the expression level of POU2AF1 in a sample.

In one embodiment, the in vitro non-invasive prognostic method comprises a step of determining the level, amount or concentration of MMP-9 in a sample, preferably the expression level of MMP-9 in a sample.

In one embodiment, the level, amount or concentration of said biomarkers may be measured by any technology well-known by a person skilled in the art. In one embodiment, the level, amount or concentration of said biomarkers may be measured at the genomic and/or transcriptomic and/or proteomic level.

In one embodiment, the level, amount or concentration of at least one biomarker selected form the group comprising or consisting of TCL1A, BLK and POU2AF1 is measured from biological sample. In one embodiment, the level, amount or concentration of at least one biomarker selected from TCL1A, BLK and POU2AF1 is measured from blood sample.

In one embodiment, the level, amount or concentration of at least one biomarker selected from the group comprising or consisting of TCL1A, BLK and POU2AF1, preferably the expression level of said biomarker is determined from whole blood or blood cells, preferably peripheral blood mononuclear cells (PBMC). Whole blood may be obtained using preservative conditions such as with PAXgene® and Tempus™ blood tubes or with collection tubes with anticoagulant agents such as sodium citrate (Citrate), ethylenediaminetetraacetic acid (EDTA), sodium fluoride (Fluoride), and sodium heparin (Heparin). Blood cells may be isolated by any technology well-known by a person skilled in the art, comprising FICOLL, magnetic microbeads and Fluorescence Activated Cell Sorting (FACS).

In one embodiment, the level, amount or concentration of at least one biomarker selected from the group comprising or consisting of TCL1A, BLK and POU2AF1, preferably the expression level of at least one biomarker selected form the group comprising or consisting of TCL1A, BLK and POU2AF1 is measured at the transcriptomic level. The transcriptomic level (corresponding to nucleic acid transcripts level) of each biomarker may be measured by any technology well-known by a person skilled in the art, comprising nucleic microarrays, quantitative, digital or real-time PCR (Polymerase Chain Reaction), High Throughput PCR/qPCR/dPCR, hybridization with target-specific probes (e.g. NanoString's nCounter® technology) and RNA-sequencing.

In one embodiment, the level, amount or concentration of MMP-9, preferably the expression level of MMP9 is measured from biological samples. In one embodiment, the level, amount or concentration of MMP9, preferably the expression level of MMP-9 is measured from blood samples. In one embodiment, the level, amount or concentration of MMP-9, preferably the expression level of said biomarker is determined from plasma and/or serum samples, preferably plasma samples. In one embodiment, the plasma samples are obtained in heparined-tubes, EDTA tubes or citrate-tubes, preferably heparined-tubes. In one embodiment, the serum samples are obtained in tubes containing no anticoagulant or preservative.

In one embodiment, the level, amount or concentration of MMP-9, preferably the expression level of MMP-9 is measured at the protein level. The protein level MMP-9 may be measured by any technology well-known by a person skilled in the art, comprising protein microarrays, enzyme-linked immunosorbent assay (ELISA), Mass spectrometry, Immunohistochemistry, Western-Blot and flow cytometry.

In one embodiment, a reduction dimension of expression (i.e. dimensionality reduction of expression) of at least two biomarkers measured in step (a) is optionally obtained. In one embodiment, a reduction dimension of the level, amount or concentration or of expression (i.e. dimensionality reduction) of at least two biomarkers selected from the group comprising or consisting of TCL1A, BLK, POU2AF1 and matrix metalloproteinase-9 (MMP-9) is obtained. In one embodiment, a reduction dimension of expression (i.e. dimensionality reduction of expression) of TCL1A, BLK and POU2AF1 is optionally obtained. In one embodiment, the reduction dimension of expression of the biomarker is obtained by any technology well-known by a person skilled in the art, comprising a principal component analysis (PCA), LDA (linear discriminant analysis), SVD (singular value decomposition), and linear regression, preferably by using a PCA.

For example, performing the dimensionality reduction of expression or of the level, amount or concentration of the biomarker using PCA may be achieved by following these steps:
collecting expression data (i.e. the level, amount or concentration of the biomarker) for the biomarker across samples (e.g. biological samples, blood samples, plasma samples);
putting expression data in one format (for example, in a table/matrix with rows representing a sample identity and a column representing the level, amount or concentration of the biomarker);
optionally, standardizing the expression data;
calculate principal components using PCA;
analyze a variance ratio of each principal component to interpret how the biomarker contributes to each principal component.

Based on the contribution of the biomarker to each principal component, it may be inferenced how the biomarker described herein above relates to a specific outcome or biological process.

In another example, performing the dimensionality reduction of expression or of the level, amount or concentration of the biomarker using LDA may be achieved by following these steps:
collecting expression data for the biomarker across samples;
putting expression data in one format;
optionally, standardizing the expression data;
calculating linear discriminants, which represent combinations of the biomarker level, amount or concentration that best discriminate between different sample groups or classes;
analyzing the linear discriminants to understand how the biomarker contributes to each discriminant.

The biomarker contribution to each discriminant may be interpreted in terms of its relevance to specific outcomes or biological processes. In another example, performing the dimensionality reduction of expression or of the level, amount or concentration of the biomarker using SVD may be achieved by following these steps:
organizing the expression data (for example expression data may be image data) into a matrix format, where each row represents a pixel and each column represents an image;
compute the Singular Value Decomposition of the image data matrix so as to obtain a decomposition of the original matrix (i.e. expression data matrix) into three matrices: U, Σ, and V^{T}. The matrix Σ contains singular values, which represent the importance of each dimension (or component) in the original dataset (i.e. the expression image data). The matrix U is a left singular matrix containing the left singular vectors of the original matrix. Each column of U represents the direction of maximum variance in the original data. The matrix V^{T} is the transpose of the right singular matrix V. It contains the right singular vectors of the original matrix. Each row of V^{T} represents a linear combination of original variables (i.e. columns of the data expression matrix) that corresponds to a singular vector;
selecting k singular values and their corresponding columns in the matrices U and V^{T}. These columns represent the most important components of the original dataset;
retain only these k components and discard the rest, effectively reducing the dimensionality of the original dataset.

To reconstruct the original dataset using the retained components, this may be achieved by multiplying the selected columns of U and V^{T} by the corresponding singular values in Σ.

The reduced dimension dataset may capture the most significant features or characteristics common to images included in the original dataset. Applying SVD for dimensionality reduction may effectively compress information contained in the original dataset while retaining its essential structure and characteristics. This reduced representation can facilitate tasks such as classification, clustering, or visualization.

In one embodiment, at least one clinical data variable is obtained. In one embodiment, the clinical data variable is a binary variable. In one embodiment, the clinical data variable is selected from the group comprising allograft rejection experience before sampling, maintenance treatment at sampling, and induction treatment at transplantation, and mathematical combinations thereof.

In the context of the invention, the allograft rejection experience before sampling refers to a subject previously diagnosed with allograft rejection. Allograft rejection can be defined by i) acute cellular rejection, diagnosed by a decrease of lung function associated with compatible pathological findings on transbronchial biopsies and/or significant improvement of lung function after appropriate treatment (high dose corticosteroids) and/or by ii) antibody mediated rejection diagnosed with all or some of the following elements: blood donor specific anti-HLA antibodies, C4d deposition on transbronchial biopsies, alteration of lung function, compatible pathological findings on transbronchial biopsies. The allograft rejection experience is a binary clinical data variable that can only take one of the two values—0 or 1—, wherein 0 indicates that the subject has no previous allograft experience of rejection and 1 indicates that the subject has at least one allograft experience of rejection.

In the context of the invention, the maintenance treatment at sampling refers to the administration to a subject of a therapeutic regimen in order to abrogate, inhibit, slow or reverse the progression of a given disease or condition, and/or to ameliorate clinical symptoms of the given disease or condition, and/or to prevent the appearance of further clinical symptoms of the given disease or condition, e.g. allograft rejection. In the context of the invention, the maintenance treatment is adapted to a subject having a lung transplant and being subjected to treatment allowing to prevent allograft rejection. Usually, drugs administrated during the maintenance treatment are immunosuppressive agents such as for example calcineurin inhibitor (e.g., cyclosporine A (CsA) and tacrolimus). During treatment of an illness, maintenance treatment is used to keep the subject in remission. The maintenance treatment at sampling is a binary clinical data variable that can only take one of the two values—0 or 1—, wherein 0 indicates that the subject did not receive drug for maintenance treatment, and 1 indicates that the subject received drug for maintenance treatment, preferably tacrolimus.

In the context of the invention, the induction treatment at transplantation refers to a treatment comprising or consisting in providing an efficient dose of drug to a subject to promote strong early immunosuppression and prevent early graft rejection for a short period of time. The dose of drug during the induction treatment correspond to a higher dose that during maintenance treatment. Usually, drug regiment being center dependent. Immunosuppressive drug may be selected from the group comprising or consisting of calcineurin inhibitor (e.g., cyclosporine or tacrolimus), nucleotide blocking agent (e.g., azathioprine or mycophenolate mofetil), tyrosine kinase inhibitors (e.g., everolimus, sirolimus) corticosteroids and specific monoclonal antibodies including basiliximab (anti-IL2R) or anti thymocyte globulin (ATG). The induction treatment at transplantation is a binary clinical data variable that can only take one of the two values —0 or 1—, wherein 0 indicates that the subject did not receive an induction treatment and 1 indicates that the subject has received an induction treatment.

In one embodiment, the in vitro non-invasive prognostic method for assessing the risk of Chronic Lung Allograft Dysfunction (CLAD) in a subject comprises mathematically combining in a multivariate model in step (c) biomarkers (i.e. biological variables) and clinical data variables as described herein above.

According to the invention, the method comprises mathematically combining in a multivariate model said at least one variable obtained in step (a) and/or a reduction dimension of expression of at least two biomarkers obtained/measured in step (a); and said at least one clinical data variable obtained in step (b).

In one embodiment, the method comprises mathematically combining in a multivariate model said at least one variable obtained in step (a) and said reduction dimension of expression of at least two biomarkers obtained/measured in step (a); and said at least one clinical data variable obtained in step (b).

According to the invention, mathematically combining in a multivariate model comprises receiving as input of said multivariate model at least the following variables:
i) the level, amount or concentration, preferably the expression level of TCL1A, BLK and POU2AF1, and the clinical data variable allograft rejection experience before sampling, or
ii) the level, amount or concentration, preferably the expression level of TCL1A, BLK and POU2AF1, and the clinical data variable maintenance treatment at sampling, or
iii) the level, amount or concentration, preferably the expression level of TCL1A, BLK, and POU2AF1, and the clinical data variable induction treatment at transplantation.

In one embodiment, the method of the invention comprises mathematically combining in a multivariate model in step (c) the level, amount or concentration, preferably the expression level of TCL1A, BLK and POU2AF1, and the clinical data variable allograft rejection experience before sampling.

In one embodiment, the method of invention comprises mathematically combining in a multivariate model in step (c) the level, amount or concentration, preferably the expression level of TCL1A, BLK and POU2AF1, and the clinical data variable maintenance treatment at sampling.

In one embodiment, the method of invention comprises mathematically combining in a multivariate model in step (c) the level, amount or concentration, preferably the expression level of TCL1A, BLK and POU2AF1, and the clinical data variable induction treatment at transplantation.

In one embodiment, the method of the invention comprises mathematically combining in a multivariate model in step (c) the level, amount or concentration, preferably the expression level of TCL1A, BLK or POU2AF1, and the clinical data variable allograft rejection experience before sampling.

In one embodiment, the method of invention comprises mathematically combining in a multivariate model in step (c) the level, amount or concentration, preferably the expression level of TCL1A, BLK or POU2AF1, and the clinical data variable maintenance treatment at sampling.

In one embodiment, the method of invention comprises mathematically combining in a multivariate model in step (c) the level, amount or concentration, preferably the expression level of TCL1A, BLK or POU2AF1, and the clinical data variable induction treatment at transplantation.

In one embodiment, the method of invention comprises mathematically combining in a multivariate model in step (c) a reduction dimension of expression or of the level, amount or concentration TCL1A, BLK and POU2AF1, and the clinical data variable allograft rejection experience before sampling.

In one embodiment, the method of invention comprises mathematically combining in a multivariate model in step (c) a reduction dimension of expression or of the level, amount or concentration (i.e. dimensionality reduction of expression) of TCL1A, BLK and POU2AF1, and the clinical data variable maintenance treatment at sampling.

In one embodiment, the method of invention comprises mathematically combining in a multivariate model in step (c) a reduction dimension of expression or of the level, amount or concentration (i.e. dimensionality reduction of expression) of TCL1A, BLK and POU2AF1, and the clinical data variable induction treatment at transplantation.

In one embodiment, the method of invention comprises mathematically combining in a multivariate model in step (c) a reduction dimension of expression or of the level, amount or concentration (i.e. dimensionality reduction of expression) of TCL1A, BLK, POU2AF1 and MMP-9, and the clinical data variable allograft rejection experience before sampling.

In one embodiment, the method of invention comprises mathematically combining in a multivariate model in step (c) a reduction dimension of expression or of the level, amount or concentration (i.e. dimensionality reduction of expression) of TCL1A, BLK, POU2AF1 and MMP-9, and the clinical data variable maintenance treatment at sampling.

In one embodiment, the method of invention comprises mathematically combining in a multivariate model in step (c) a reduction dimension of expression or of the level, amount or concentration (i.e. dimensionality reduction of expression) of TCL1A, BLK, POU2AF1 and MMP-9, and the clinical data variable induction treatment at transplantation.

In one embodiment, the method of invention comprises mathematically combining in a multivariate model in step (c) the measured of the level, amount or concentration, preferably the expression level of the biomarker MMP-9 obtained in step (a); a reduction dimension of expression or of the level, amount or concentration (i.e. dimensionality reduction of expression) of the biomarkers TCL1A, BLK and POU2AF1; and the obtained clinical data variable allograft rejection experience before sampling obtained in step (b).

In one embodiment, the method of invention comprises mathematically combining in a multivariate model in step (c) the measured of the level, amount or concentration, preferably the expression level of the biomarker MMP-9 obtained in step (a); a reduction dimension of expression or of the level, amount or concentration (i.e. dimensionality reduction of expression) of the biomarkers TCL1A, BLK and POU2AF1; and the obtained clinical data variable maintenance treatment at sampling obtained in step (b).

In one embodiment, the method of invention comprises mathematically combining in a multivariate model in step (c) the measured of the level, amount or concentration, preferably the expression level of the biomarker MMP-9 obtained in step (a); a reduction dimension of expression or of the level, amount or concentration (i.e. dimensionality reduction of expression) of the biomarkers TCL1A, BLK and POU2AF1; and the obtained clinical data variable induction treatment at transplantation obtained in step (b).

In one embodiment, the method of invention comprises mathematically combining in a multivariate model in step (c) the measured of the level, amount or concentration, preferably the expression level of the biomarker MMP-9 obtained in step (a); and the obtained clinical data variable allograft rejection experience before sampling obtained in step (b).

In one embodiment, the method of invention comprises mathematically combining in a multivariate model in step (c) the measured of the level, amount or concentration, preferably the expression level of the biomarker MMP-9 obtained in step (a); and the obtained clinical data variable maintenance treatment at sampling obtained in step (b).

In one embodiment, the method of invention comprises mathematically combining in a multivariate model in step (c) the measured of the level, amount or concentration, preferably the expression level of the biomarker MMP-9 obtained in step (a); and the obtained clinical data variable induction treatment at transplantation obtained in step (b).

In one embodiment, the multivariate model is obtained from a Cox model more precisely a multivariate Cox regression with stepwise selection based on Akaike information criterion (AIC). This multivariate Cox regression with stepwise selection based on AIC extends the traditional Cox model by allowing for the inclusion of multiple predictor variables and employs a stepwise selection procedure guided by the Akaike information criterion to identify the most relevant variables for predicting survival outcomes. In one embodiment, the multivariate Cox model is time-fixed or time-dependent, preferably is time-dependent. The multivariate model is time-fixed whenever all the combined variables in the multivariate model do not dependent on time; in other words, the contribution of each variable of the combined variables does not change with time. In contrast, the multivariate model is time-dependent whenever at least one of the combined variables is time-dependent; in other words, the contribution of at least one variable of the combined variables changes with time. The advantage of using a time-dependent multivariate Cox model lies in its ability to capture changes in the effect of predictor variables over time.

A Cox model may be trained by estimating the relationship between covariates (predictor variables) a hazard function (e.g. exponential, weibull, logistic), which represents the instantaneous rate of occurrence of an event over time. The Cox model focuses on estimating a hazard ratio, which quantifies a risk of experiencing an event (such as the risk of CLAD) for different levels of a predictor variable. The Cox model may be trained using a maximum likelihood estimation. In this process, the model parameters, including the coefficients for each predictor variable, are estimated to maximize the likelihood of observing the actual survival times given the predictor variables and the censoring information. This involves iteratively adjusting the parameter estimates until they converge to values that optimize the likelihood function. The model is fitted to the data by comparing the observed survival times and censoring indicators with the predicted hazard function generated by the model. The algorithm iteratively adjusts the model parameters until the likelihood of observing the data is maximized, indicating the best fit of the model to the data.

In one embodiment, the method of the invention comprises a step of obtaining a risk score to assess the risk of CLAD in the subject.

According to the invention, the risk score is calculated according to the linear combination component of the multivariate Cox model multiplied by their regression coefficient (β). The risk score can be written as the following formula: risk score=Σ(βᵢ×Variableᵢ)+β₀ where βᵢ represents the coefficients for each variable (also called predictor or predictor variable) and “β₀” represents the intercept of the equation.

In one embodiment, the method of the invention comprises a step of determining a risk of having CLAD for the subject based on the obtained risk score. In one embodiment, step of determining a risk of having CLAD for the subject comprises comparing the risk score obtained for the subject with a reference risk score. In one embodiment, the reference risk score corresponds to a threshold obtained by comparing a score from a reference subject being identified as not at risk of developing CLAD and a score from a subject being identified as at risk of developing CLAD. In one embodiment, the reference risk score may be a threshold.

In one embodiment, the reference risk score may be derived from transplanted subjects having CLAD. In one embodiment, the reference risk score may be derived from “stable” transplanted subjects who does not have CLAD. In one embodiment, the reference risk score may be derived from transplanted subjects having CLAD and from “stable” transplanted subjects who does not have CLAD.

In some embodiments, at step d) of the method, the subject is determined at low risk or at high risk of developing CLAD, depending on the obtained risk score.

In one embodiment, the reference risk is a threshold distinguishing “stable” transplanted subject and transplanted subject at risk of CLAD. In one embodiment, if a risk score obtained from a subject is superior to the reference risk score (corresponding to the score obtained for a reference subject), then the subject is at risk of CLAD. In one embodiment, if a risk score obtained from a subject is inferior to the reference risk score, then the subject is considered as having low risk of developing CLAD. In one embodiment, if a risk score obtained from a subject is superior or equal to the reference risk score, then the subject is considered as at risk of developing CLAD.

In one embodiment, the subject is at risk of developing a Chronic Lung Allograft Dysfunction (CLAD), when the obtained risk score at step (d) is superior or equal to 0.70, superior or equal to 0.75, preferably superior or equal to 0,80, more preferably superior or equal to 0,85, such as superior or equal to 0.89, superior or equal to 0.91, 0.92, 0,93, 0.94, 0.95, 0.96, 0.97 and even more.

In one embodiment, the subject is at low risk of developing a Chronic Lung Allograft Dysfunction (CLAD), when the obtained risk score at step (d) is inferior or equal to 0.70, inferior or equal to 0.75, preferably inferior or equal to 0,80, more preferably inferior or equal to 0,85, such as inferior or equal to 0.89, inferior or equal to 0.91, 0.92, 0,93, 0.94, 0.95, 0.96, 0.97.

In one embodiment, a risk score obtained for the subject which is different from the reference risk score is indicative of a risk of CLAD. Preferably, in this embodiment, the reference risk score is derived from stable transplanted subject who does not have CLAD.

In one embodiment, a risk score obtained for the subject which is not different from the reference risk score is indicative of a risk of CLAD. Preferably, in this embodiment, the reference risk score is derived from transplanted subject having CLAD.

In one embodiment, a risk score from the subject is considered different from a reference risk score if the risk score from the subject is superior by about 1%, 2%, 3%, 4%, 5%, 6%, 7%, 8%, 9%, 10%, 20%, 30%, 40%, 50%, 60%, 70%, 80%, 90%, 100% or more compared to the reference prognostic (i.e. reference risk score or threshold).

In one embodiment, the risk score may be used to predict the risk of CLAD in a subject, including BOS and RAS.

In one embodiment, the risk score is suitable to predict the risk of having a bronchiolitis obliterans syndrome (BOS). In other words, the risk score may be used to predict the risk of having a BOS.

In one embodiment, the risk score is suitable to predict the risk of having a restrictive allograft syndrome (RAS). In other words, the risk score may be used to predict the risk of having a RAS.

In one embodiment, the in vitro non-invasive prognostic method being computer implemented.

The present invention further relates to a method for preventing the risk of having CLAD in a subject, comprising
a. a first step consisting in implementing the in vitro non-invasive prognostic method, so as to obtain a risk score,
b. determining a risk of having CLAD for the subject based on the obtained risk score, and
c. determining the personalized course of treatment for the subject based on the obtained risk score.

In one embodiment, when it is concluded at step b) that the subject is at risk of having CLAD, the subject is thus eligible to preventive or therapeutic treatment.

The present invention further relates to a method for preventing the risk of having CLAD in a subject, comprising:
obtaining a risk of CLAD using a method of the invention, and
determining a personalized course of treatment for the subject based on the obtained risk score.

In one embodiment, when it is concluded that the subject is at risk of having CLAD, the subject is thus eligible to preventive or therapeutic treatment.

In one embodiment, the preventive or therapeutic treatment is selected from the group comprising: immunosuppressive and immunomodulatory therapies drugs, anti-fibrotic treatment, cellular-based therapies, such as the use of mesenchymal stromal cells, endothelial progenitor cells, regulatory T cells (Tregs) or chimeric antigen receptors (CAR) Tregs, prophylactic treatment with azithromycin, total lymphoid irradiation (TLI) or extracorporeal photopheresis (ECP), current ant novel biotherapy such as anti-TNFα, anti-IL6R or JAKs inhibitors.

As for example, the immunosuppressive and immunomodulatory therapies drugs may be selected from the group comprising: cyclosporine A, tacrolimus, azathioprine, methotrexate, rapamycin, mycophenolate mofetil, mycophenolic acid, mycophenolate sodium, 6-mercaptopurine, 6-thioguanine, rituximab, sirolimus, everolimus, basiliximab, daclizumab, belatacept, alemtuzumab, anti-thymocyte globulin (ATG) corticosteroids, or any combination thereof.

In one embodiment, when it is concluded at step b) that the subject is not at risk of having CLAD, the subject is thus eligible to for immunosuppressive therapy minimization and lower frequency of clinical follow-up. “Immunosuppressive therapy minimization” refers to the progressive reduction of an immunosuppressive therapy.

The present invention further relates to a method for prevent CLAD in a subject comprising:
1) determining a subject as being at risk of having CLAD by:
   a. obtaining at least one variable from the subject by measuring the the level, amount or concentration, preferably the expression level of at least one biomarker in a biological sample from the subject, wherein said at least one biomarker is selected from the group comprising or consisting of TCL1A, BLK, POU2AF1 and matrix metalloproteinase-9 (MMP-9),
      a1. optionally mathematically obtaining a reduction dimension of expression of at least two of the biomarkers measured in step (a),
   b. obtaining at least one clinical data variable selected from the group comprising allograft rejection experience before sampling, maintenance treatment at sampling, induction treatment at transplantation, and mathematical combinations thereof, and
   c. mathematically combining in a multivariate model
      i. said at least one variable obtained in step (a) and/or said reduction dimension of expression of the at least two biomarkers when obtained in step (a1.); and
      ii. said at least one clinical data variable obtained in step (c);
   to obtain a risk score thereby determining the risk of CLAD in the subject; and
2) implementing an adapted patient care depending on the risk of CLAD comprising: administering at least one immunosuppressive and immunomodulatory therapies selected from the group comprising immunosuppressive and immunomodulatory drugs, anti-fibrotic treatment, cellular-based therapies, such as the use of mesenchymal stromal cells, endothelial progenitor cells, regulatory T cells (Tregs) or chimeric antigen receptors (CAR) Tregs, prophylactic treatment with azithromycin, total lymphoid irradiation (TLI) or extracorporeal photopheresis (ECP), current ant novel biotherapy such as anti-TNFα, anti-IL6R or JAKs inhibitors.

The present invention further relates to a method for prevent CLAD in a subject comprising:
1) determining a subject as being at risk of having CLAD by:
   a. obtaining at least one variable from the subject by measuring the level, amount or concentration, preferably the expression level of at least one biomarker in a biological sample from the subject, wherein said at least one biomarker is selected from TCL1A, BLK, POU2AF1 and matrix metalloproteinase-9 (MMP-9),
   b. obtaining at least one clinical data variable selected from a group comprising allograft rejection experience before sampling, maintenance treatment at sampling, and induction treatment at transplantation, and mathematical combinations thereof,
   c. obtaining a risk score by mathematically combining in a multivariate model:
      i. said at least one variable obtained in step (a) and/or a reduction dimension of the level, amount or concentration mathematically obtained from at least two biomarkers measured in step (a); and said at least one clinical data variable obtained in step (b); and
      ii. determining the risk of Chronic Lung Allograft Dysfunction for the subject by comparing the obtained risk score with a reference risk score; and
2) implementing an adapted patient care depending on the risk of CLAD comprising: administering at least one immunosuppressive and immunomodulatory therapies selected from the group comprising immunosuppressive and immunomodulatory drugs, anti-fibrotic treatment, cellular-based therapies, such as the use of mesenchymal stromal cells, endothelial progenitor cells, regulatory T cells (Tregs) or chimeric antigen receptors (CAR) Tregs, prophylactic treatment with azithromycin, total lymphoid irradiation (TLI) or extracorporeal photopheresis (ECP), current ant novel biotherapy such as anti-TNFα, anti-IL6R or JAKs inhibitors.

In one embodiment, the method for prevent CLAD in a subject further comprises determining in step 1) a subject as being at risk of having BOS and/or RAS.

In one embodiment, determining a subject as being at risk of having CLAD comprises mathematically combining in a multivariate model said at least one variable obtained in step (a) or said reduction dimension of expression or of the level, amount or concentration of the biomarkers obtained in step (a1); and said at least one clinical data variable obtained in step (b).

In one embodiment, determining a subject as being at risk of having CLAD comprises mathematically combining in a multivariate model said at least one variable obtained in step (a) and/or a reduction dimension of the level, amount or concentration mathematically obtained from at least two biomarkers measured in step (a); and said at least one clinical data variable obtained in step (b).

In one embodiment, determining a subject as being at risk of having CLAD comprises mathematically combining in a multivariate model in step (c) at least the following variables:
the level, amount or concentration, preferably the expression level of TCL1A, BLK, and POU2AF1, and the clinical data variable allograft rejection experience before sampling,
the level, amount or concentration, preferably the expression level of TCL1A, BLK, and POU2AF1, and the clinical data variable maintenance treatment at sampling,
the level, amount or concentration, preferably the expression level of TCL1A, BLK, and POU2AF1, and the clinical data variable induction treatment at transplantation,
a reduction dimension of expression or of the level, amount or concentration (i.e. dimensionality reduction of expression) of TCL1A, BLK, and POU2AF1, and the clinical data variable allograft rejection experience before sampling,
a reduction dimension of expression or of the level, amount or concentration (i.e. dimensionality reduction of expression) of TCL1A, BLK, and POU2AF1, and the clinical data variable maintenance treatment at sampling,
a reduction dimension of expression or of the level, amount or concentration of (i.e. dimensionality reduction of expression) TCL1A, BLK, and POU2AF1, and the clinical data variable induction treatment at transplantation.
a reduction dimension of expression or of the level, amount or concentration (i.e. dimensionality reduction of expression) of TCL1A, BLK, POU2AF1, and MMP-9, and the clinical data variable allograft rejection experience before sampling,
a reduction dimension of expression or of the level, amount or concentration (i.e. dimensionality reduction of expression) of TCL1A, BLK, POU2AF1, and MMP-9, and the clinical data variable maintenance treatment at sampling,
a reduction dimension of expression or of the level, amount or concentration of TCL1A, BLK, POU2AF1, and MMP-9 and the clinical data variable induction treatment at transplantation,
MMP-9 expression; a reduction dimension of expression or of the level, amount or concentration (i.e. dimensionality reduction of expression) of TCL1A, BLK, POU2AF1, and the clinical data variable allograft rejection experience before sampling,
MMP-9 expression; a reduction dimension of expression or of the level, amount or concentration (i.e. dimensionality reduction of expression) of TCL1A, BLK, POU2AF1, and the clinical data variable maintenance treatment at sampling,
MMP-9 expression, a reduction dimension of expression or of the level, amount or concentration (i.e. dimensionality reduction of expression) of TCL1A, BLK, POU2AF1, and the clinical data variable induction treatment at transplantation,
the level, amount or concentration of MMP-9 and the clinical data variable allograft rejection experience before sampling,
the level, amount or concentration, preferably the expression level of MMP-9 and the clinical data variable maintenance treatment at sampling,
the level, amount or concentration, preferably the expression level of MMP-9 and the clinical data variable induction treatment at transplantation,
the level, amount or concentration, preferably the expression level of TCL1A and the clinical data variable allograft rejection experience before sampling,
the level, amount or concentration, preferably the expression level of TCL1A and the clinical data variable maintenance treatment at sampling,
the level, amount or concentration, preferably the expression level of TCL1A and the clinical data variable induction treatment at transplantation,
the level, amount or concentration, preferably the expression level of BLK and the clinical data variable allograft rejection experience before sampling,
the level, amount or concentration, preferably the expression level of BLK and the clinical data variable maintenance treatment at sampling,
the level, amount or concentration, preferably the expression level of BLK and the clinical data variable induction treatment at transplantation,
the level, amount or concentration, preferably the expression level of POU2AF1 and the clinical data variable allograft rejection experience before sampling,
the level, amount or concentration, preferably the expression level of POU2AF1 and the clinical data variable maintenance treatment at sampling, or
the level, amount or concentration, preferably the expression level of POU2AF1 and the clinical data variable induction treatment at transplantation.

In one embodiment, determining a subject as being at risk of having CLAD comprises a step of obtaining mathematically a risk score.

The present invention also relates to a kit-of-parts for performing the method disclosed herein, comprising:
means for determining the level, amount or concentration, preferably the expression level of at least one biomarker selected from the group comprising or consisting of TCL1A, BLK, POU2AF1, and MMP-9, and
a device such as for example a program to receive/obtained input corresponding to the at least one clinical data variable.

In one embodiment, the kit-of-parts further comprises instructions for use to perform the method.

The present invention also relates to a computer system for assessing a risk of Chronic Lung Allograft Dysfunction (CLAD) in a subject.

As used herein, the term “computer system” refers to any and all devices capable of storing and processing information and/or capable of using the stored information to control the behavior or execution of the device itself, regardless of whether such devices are electronic, mechanical, logical, or virtual in nature.

In one embodiment, the computer system comprises:
i) at least one processor, and
ii) at least one computer readable storage medium that at least one code readable by the processor, when executed but the processor, causes the processor to:
   a. receive (i) input levels, amounts or concentrations of at least one biomarker, wherein said at least one biomarker is selected from the group comprising or consisting of TCL1A, BLK, POU2AF1 and matrix metalloproteinase-9 (MMP-9), and (ii) input values of at least one clinical data variable selected from a group comprising allograft rejection experience, maintenance treatment at sampling, and induction treatment at transplantation, and mathematical combinations thereof,
   b. analyze and transform the input levels, amounts or concentrations and the input values, to derive a risk score,
   c. generate an output, wherein the output is the risk score, and
   d. provide the risk of Chronic Lung Allograft Dysfunction for the subject by comparing the obtained risk score with a reference risk score.

As used herein, the term “processor” is meant to include any integrated circuit or other electronic device capable of performing an operation on at least one instruction word, such as, e.g., executing instructions, codes, computer programs, and scripts which it accesses from a storage medium. However, the term “processor” should not be construed to be restricted to hardware capable of executing software, and refers in a general way to a processing device, which can for example include a computer, a microprocessor, an integrated circuit, or a programmable logic device (PLD). The processor may also encompass one or more graphics processing units (GPU), whether exploited for computer graphics and image processing or other functions. Additionally, the instructions and/or data enabling to perform associated and/or resulting functionalities may be stored on any processor readable medium, including, but not limited to, an integrated circuit, a hard disk, a magnetic tape (including floppy disk and zip diskette), an optical disc (including Blu ray, compact disc and digital versatile disc), a flash memory (including memory card and USB flash drive) a random access memory (RAM) (including dynamic and static RAM), a read only memory (ROM) or a cache. Instructions may be in particular stored in hardware, software, firmware or in any combination thereof.

### BRIEF DESCRIPTION OF THE DRAWINGS

FIGS. 1 A-D are a combination of graphs showing the relative expression of the biomarkers determined by qPCR between stable and BOS patients.

FIG. 1A shows the relative expression of BLK between stable and BOS patients. FIG. 1B shows the relative expression of POU2AF1 between stable and BOS patients. FIG. 1C shows the relative expression of TCL1A between stable and BOS patients. FIG. 1D shows the correlation between the relative expression of the 3 biomarkers.

FIGS. 2 A-C are a combination of graphs showing the dimension reduction of the expression of BLK, POU2AF1, TCL1A using Principal component analysis (PCA). FIG. 2A shows a PCA score plot indicating the expression differences of the 3 biomarkers between stable and BOS patients. FIG. 2B shows statistical comparison of PCA1 score between stable and BOS patients. FIG. 2C shows the correlation between PCA1 score and mean of the 3 biomarkers expression values.

FIG. 3 shows time dependent ROC curves. AUC of the “Risk score”, comprising the PCA1 of the 3 biomarkers and the clinical data variable “allograft rejection experience before sampling” (Pred-ARE) is compared with AUC of the clinical data variables: “induction treatment at transplantation”, “maintenance treatment at sampling” and “FEV1”.

FIGS. 4A-C are a combination of graphs showing the risk score values of CLAD calculated from the PCA1 of the 3 biomarkers and “allograft rejection experience before sampling” (Pred-ARE) clinical parameter. FIG. 4A compares the risk score values between stable and BOS patients after 24 months post-transplantation. FIG. 4B compares the risk score values between stable, BOS and RAS patients after the 18- and 24-months post-transplantation. FIG. 4C compares the risk score values between stable and CLAD (BOS+RAS) patients after the 18- and 24-months post-transplantation.

FIGS. 5A-B are a combination of graphs showing the plasmatic dosage of MMP-9 realized before the graft, 1 year after the graft and 2 years after the graft. The subjects are classified according to CLAD occurrence: Stable (did not developed CLAD), CLAD (who developed CLAD). FIG. 5A shows the comparison of plasmatic dosage of MMP-9 between subjects who did not developed CAD within 3 years post-transplant and subjects who developed CAD within 3 years post-transplant. FIG. 5B shows the comparison of plasmatic dosage of MMP-9 between subjects who did not develop CLAD within 5 years post-transplant and subjects who developed CAD within 5 years post-transplant.

FIG. 6 shows the predictive performance of MMP-9 by Precision-Recall curve.

FIGS. 7 A-C are a combination of graphs showing time dependent ROC curves. FIG. 7A shows AUC of model (i.e. Risk score) combining the expression of POU2AF1 with the clinical data variable “allograft rejection experience before sampling” (Pred-ARE). FIG. 7B shows AUC of model (i.e. Risk score) combining the expression of TCL1A with the clinical data variable “allograft rejection experience before sampling” (Pred-ARE). FIG. 7C shows AUC of model (i.e. Risk score) combining the expression of BLK with the clinical data variable “allograft rejection experience before sampling” (Pred-ARE).

### EXAMPLES

The present invention is further illustrated by the following examples.

### Example 1

### Materials and Methods

### Patients

Lung transplant recipients were recruited within the multicenter COLT cohort (Cohort Of Lung Transplantation; NCT00980967). The local ethical committee (Comité de Protection des Personnes Ouest 1-Tours, 2009-A00036-51) approved the study and all participants provided written informed consent. Written consent was obtained from all patients. The clinical and research activities being reported are consistent with the Principles of the Declarations of Istanbul and Helsinki and in line with the good practice recommendations of the University Hospital of Nantes.

Within the COLT cohort, recruited patients are regularly phenotyped by a blind adjudication committee as described previously, based upon pulmonary function tests and chest imaging according to ISHLT/ERS/ATS guidelines (Meyer et al, Eur Respir J 2014; 44(6): 1479-503 and Verleden et al, J Heart Transplant, 2004; 33(2): 127-33). Assessed pulmonary tests were performed before the transplantation, the day of the transplantation, one and six months after the transplantation and every six months thereafter up to 3 years post-transplantation.

### RNA Isolation

Peripheral blood samples were collected in PAXgene tubes (PreAnalytix, Qiagen, Hilden, Germany) and stored at −80 °C. Total RNA extraction and purification from peripheral blood were performed using the PAXgene Blood miRNA Kit (Qiagen, Hilden, Germany, #763134), with an on-column DNase digestion protocol, according to the manufacturer's protocol in the CRB of Nantes University Hospital. Quantity and quality of total RNA were determined using a 2100 Bioanalyzer (Agilent Technologies, Palo Alto, CA, USA), and RNA samples with an RNA integrity number above 5 were selected for further analyses.

Gene expression was assessed using quantitative PCR (qPCR). cDNA was synthesized from 500 ng of total RNA using the Superscript III (Invitrogen, Carlsbad, CA, USA). Gene expression was assessed on a Taqman StepOne plus real time PCR system (Applied Biosystems, Foster City, CA, USA) using commercially available primers: HPRT1 (Hs99999909_m1), β2M (Hs00984230_m1), ACTB (Hs99999903_m1), TCL1A (Hs00951350_m1), POU2AF1 (Hs01573371_m1) and BLK (Hs01017452_m1). Samples were run in duplicate, and the geometric mean of quantification cycle values for HPRT1, β2M, and ACTB was used for normalization. Relative expression between a sample and a reference was calculated according to the 2^{-ΔΔCt} method. The −ΔΔCq values were used for qPCR analyses. Reduction dimension of expression (i.e. dimensionality reduction of expression) of the 3 genes was performed using a principal component analysis (PCA) using the built-in R function prcomp( ) and coordinates for individuals were extracted using the factoextra R package.

### Statistical Analysis

Group comparisons were performed using nonparametric Mann-Whitney tests and Kruskal-Wallis with Dunn's post-hoc tests for two and multigroup comparisons, respectively, for continuous variables and using the chi-square test or Fisher's exact test for categorical variables. Spearman correlation was used to assess the relationship between continuous data. Time-to-event analysis was performed using univariate Cox proportional analysis between variables and time to BOS with the R survival package (Coxph function). For the constitution of the risk score, survival-associated variables selected in the univariate Cox analysis were selected using stepwise regression model comparison with the Akaike information criterion (AIC). The risk score wax calculated according to the linear combination component of the multivariate Cox model multiplied by their regression coefficient (β). The time-dependent area under the receiver operating characteristic (ROC) curve (AUC) was used to evaluate model performance (timeROC R package). Analyses were performed using R 4.0.3., FaDA and GraphPad Prism v.9 (GraphPad Software, La Jolla, CA, USA).

### Results

### Validation of BLK, POU2AF1 and TCL1A as Predictive Biomarkers of CLAD

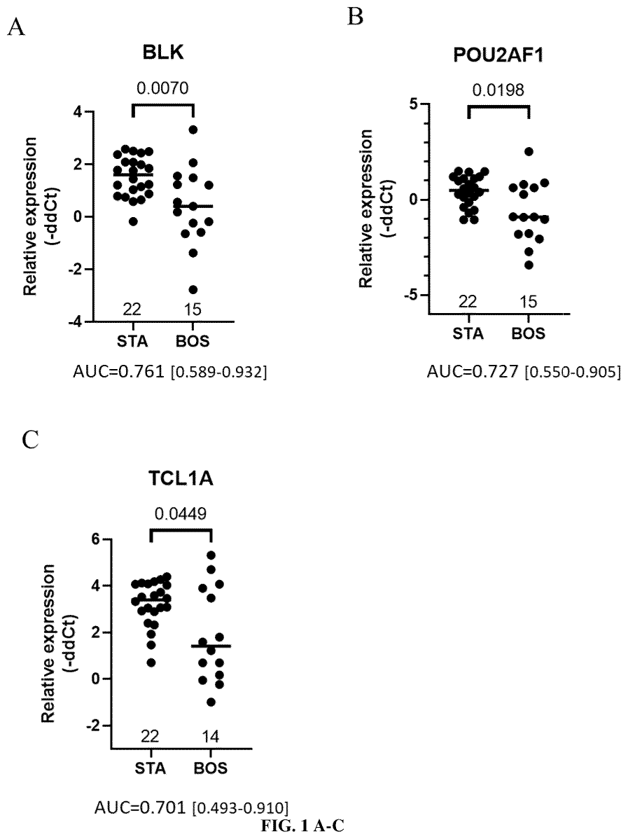

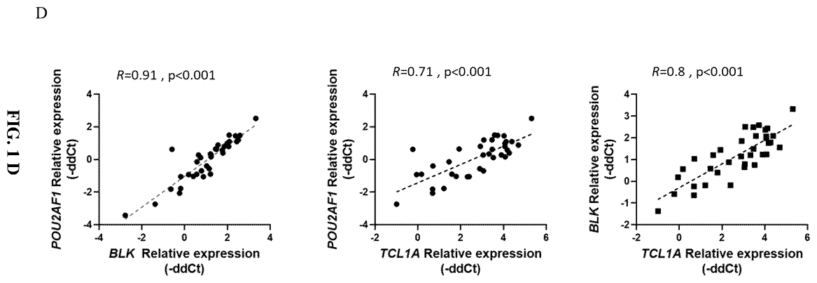

To validate that BLK, POU2AF1 and TCL1A blood expression are associated with CLAD, the expression of the 3 biomarkers was measured by quantitative PCR at 18 and 24 months after transplantation in the blood samples of lung transplanted patients of the multicenter COLT cohort. Theses samples include samples from patients with BOS and patients with good graft function (stable) at least 5 years after transplantation. As shown in FIG. 1 A-C, the expression of the 3 biomarkers measured at 24 months post-transplantation was significantly decreased (p-value<0.05) in blood from patients with CLAD occurring between 3 and 24 months after sample collection compared to stable patients. ROC analysis indicated that BLK expression discriminated well stable from BOS patients with an AUC of 0.761 [0.589-0.932](FIG. 1A). As for BLK expression, POU2AF1 and TCL1A expression discriminated well stable from BOS patients with respectively an AUC of 0.727 [0.550-0.905] and an AUC of 0.701 [0.493-0.910]. In addition, as shown in FIG. 1D, the relative expression of the 3 biomarkers are highly correlated (R>0.7, p<0.001).

These results confirm the ability of these 3 biomarkers to predict the development of CLAD in lung transplantation.

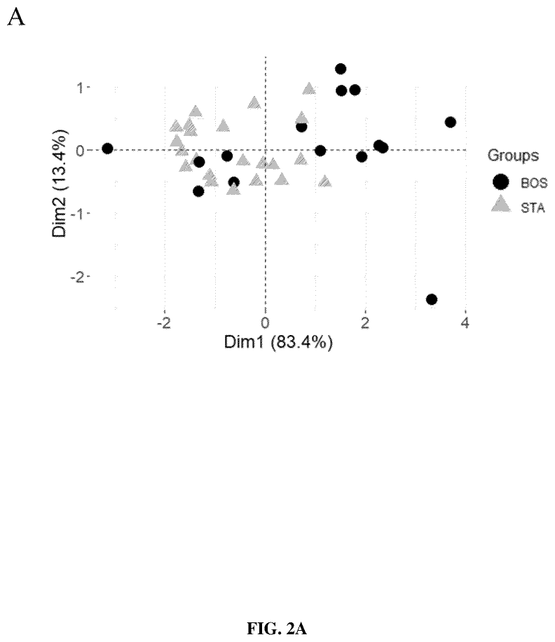

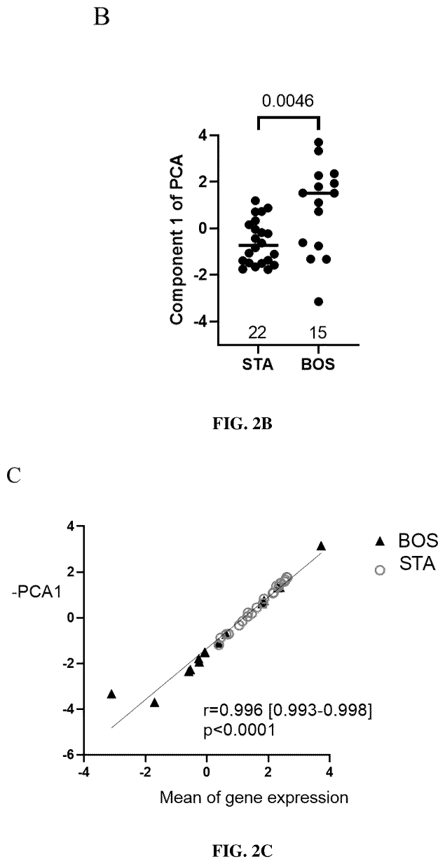

Since the 3 biomarkers are highly correlated, their expressions were resumed in the first component of a principal component analysis (PCA1, accounting for 83.4% of gene expressions variability) (FIG. 2A). PCA1 score was significantly increased (p-value<0.01) in patients with CLAD to stable patients (FIG. 2B). Finally FIG. 2C, shows a correlation between PCA1 score and mean of the 3 biomarkers expression values showing PCA resumes information from the three genes.

Identification of Clinical Parameters Associated with CLAD

In addition, to the 3 biomarkers, 3 clinical parameters associated with BOS are identified in univariate analysis at 24 months post-transplantation: experience of rejection before sampling (Pred-ARE), maintenance treatment at sampling (CNI_V6), and administration of induction treatment at transplantation (Induction) (p=0.0192, 0.01 and 0.068, respectively) (Table 1).

TABLE 1

| Characteristic | HR¹ | 95% Cl¹ | p-value |
|---|---|---|---|
| Previous ARE | 5.22 | 1.46, 18.6 | 0.011 |
| Induction (Y/N) | 0.38 | 0.14, 1.07 | 0.068 |
| CNI_V6 (Tacro/Ciclo) | 0.21 | 0.06, 0.69 | 0.01 |

¹HR = Hazard Ratio,
CI = Confidence Interval

### Identification of a Risk Score of Having CLAD

From these 3 clinical parameters and the PCA1 of the 3 biomarkers, a composite and predictive score of BOS was built using a multivariate Cox regression with stepwise selection based on AIC to select only 2 parameters.

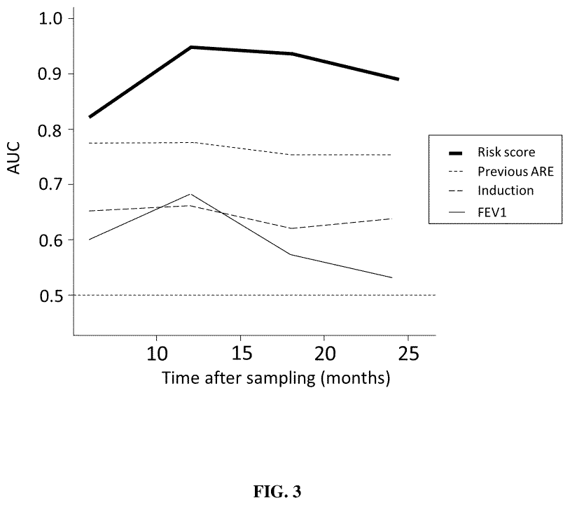

As shown in FIG. 3, the PCA1 and the experience of rejection before sampling named “Risk score” values were integrated in a time-dependent Cox model whose prediction reaches AUC superior to 0.80 at 6, 12 and 18 months after the 24 post-transplantation measure (Table 2). Time dependent ROC curves exhibited higher values from the risk score (PCA1 and the experience of rejection before sampling) compared to the measure of pulmonary function (FEV1 value compared to baseline), the experience of rejection before sampling, or administration of induction treatment.

TABLE 2
showing AUCs according times after sampling:

| Time after sampling | | | | |
|---|---|---|---|---|
| (months): | 6 | 12 | 18 | 24 |
| FEV₁ | 0.601 | 0.683 | 0.574 | 0.531 |
| Previous ARE | 0.774 | 0.776 | 0.754 | 0.751 |
| PC1A | 0.672 | 0.852 | 0.858 | 0.796 |
| Risk Score | 0.823 | 0.948 | 0.937 | 0.893 |
| Maintenance treatment | 0.718 | 0.628 | 0.629 | 0.616 |
| (Tacrolimus Uptake) | | | | |

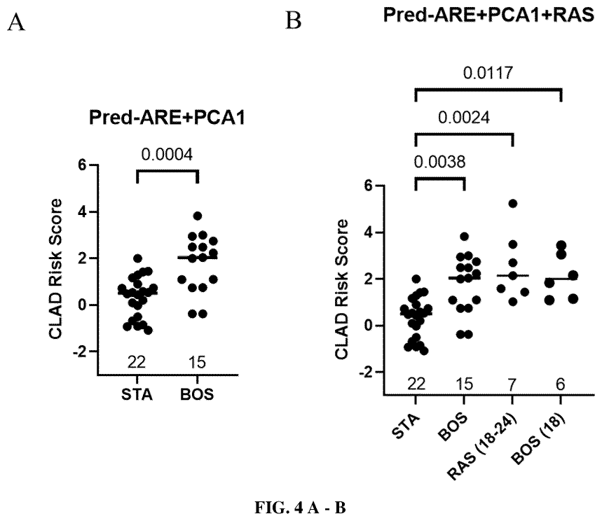

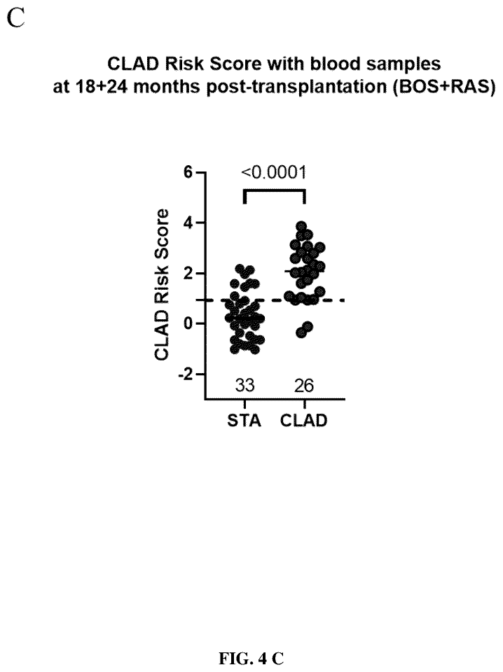

As shown in FIGS. 4A-C, the prediction/risk scores establish using the risk score with PCA1 and allograft experience of rejection before sampling are significantly increased in BOS and RAS patients compared to stable patients. Similar results are obtained when sampling was performed at 18 or 24 months after transplantation.

Moreover, models with the PCA1 and maintenance treatment at sampling or induction treatment also reaches good predictive abilities, with AUCs of 0.952 and 0.896 on year after sampling, respectively, suggesting each of these clinical parameters could be used in combination with PCA1 of the three genes.

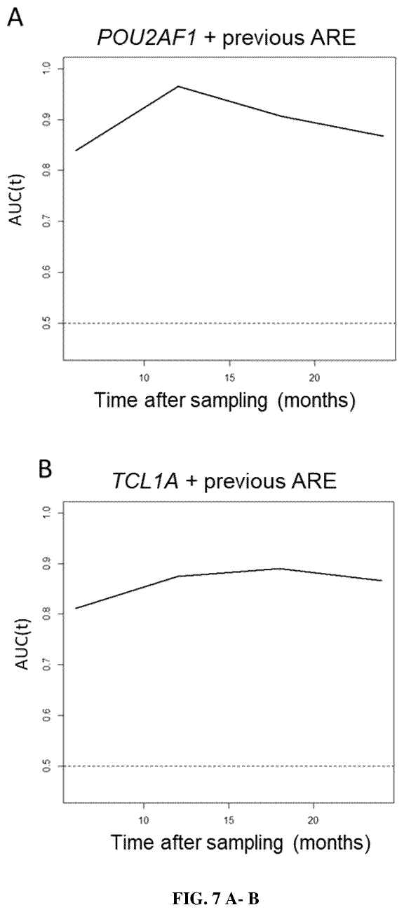

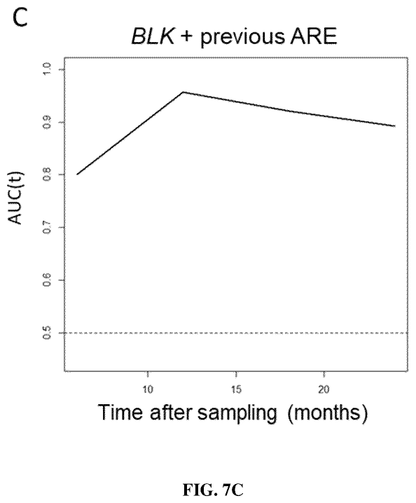

As shown in FIGS. 7 A-C, models combining the expression of each 3 genes independently (BLK, POU2AF1 or TCL1A) with the clinical data variable “allograft rejection experience before sampling” (Pred-ARE) reaches also good predictive abilities, with AUCs>0.80 for each gene.

The combination of these genes (BLK, POU2AF1 and/or TCL1A) with clinical parameter in a risk score identifies patients likely to develop CLAD and to benefit from therapy to prevent development of the pathology.

### Example 2

### Materials and Methods

### Patients

As described herein above, lung transplant recipients (LTRs) were recruited within the multicenter Cohort Of Lung Transplantation [COLT], NCT00980967) study (Comité de Protection des Personnes Ouest 1-Tours, 2009-A00036-51).

Every participant's clinical phenotype was adjudicated when follow-up reached 3-year post transplantation (3-year phenotype) and five-year post transplantation (5-year phenotype).

LTRs with a diagnosis of CLAD (BOS, RAS and Mixed) for whom plasma samples were available before transplantation, at one-year post transplantation and/or at two-year post-transplantation are selected. Patients already analysed in our previous study and those who developed CLAD before 1-year post transplantation are excluded.

### MMP-9 Detection

All plasma samples were obtained in heparined tubes, stored at −80 °C at Nantes University Hospital Biological Resource Center and never thawed before use. MMP-9 concentrations were determined with the quantitative enzyme-linked immunosorbent assay MMP-9 Human ELISA Kit (Thermo Fisher Scientific, BMS2016-2) according to the manufacturer's instruction.

### Statistical Analysis

For descriptive analysis, normally distributed continuous variables were summarized by mean plus standard deviation, whereas non-normally distributed continuous variables were summarized using median and interquartile range. Categorical variables were presented with effective and percentage of modalities. Usual statistical tests were use to compare distributions between two groups (Student's t-test, Mann-Whitney-Wilcoxon test and Fisher exact test, respectively). Missing values were systematically presented. No imputation was performed.

To assess whether MMP-9 could be predictive of CLAD, MMP-9 distributions between groups (CLAD vs Stable) were compared according to patients' follow-up using boxplots. In addition, the precision-recall area under the curve metric were computed to assess if MMP-9 concentrations could be relevant in predictive modelling CLAD in these patients. The confidence interval of the PR-AUC was computed using 1000 bootstrap iterations. Analyses were performed using R package version 4.2.2, especially using tidyverse package for data manipulation and visualization, and also tidymodels for predictive modelling.

### Results

### Higher MMP-9 Levels at 2-Year Post Transplantation for 3-Year CLAD Recipients

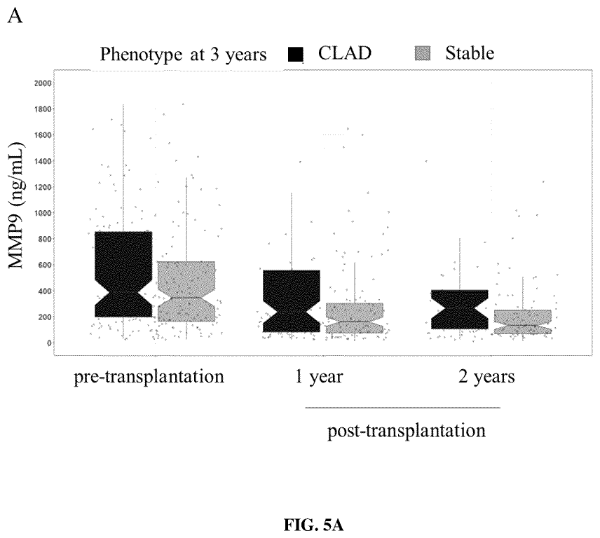

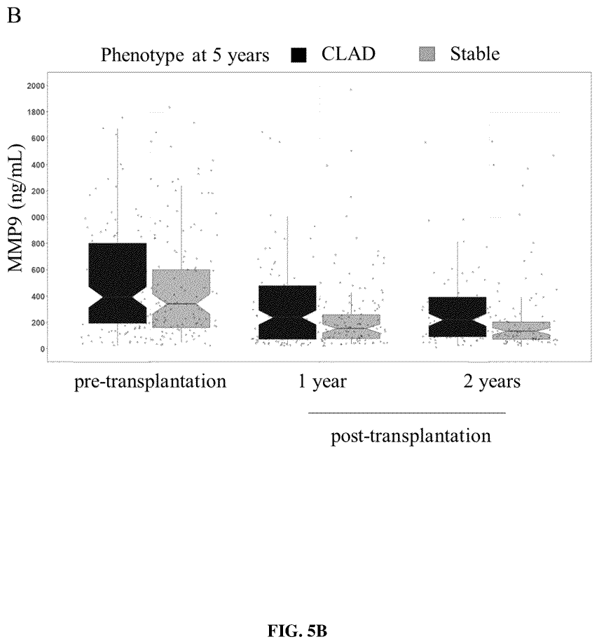

First, MMP-9 blood concentrations were determined and compared between CLAD and Stable recipients according to 3-year phenotype. Before transplantation there was no difference in MMP-9 blood levels between recipients who developed CLAD within 3 years post transplantation and those who remained stable (379 vs 378 ng/m) (Table 3). MMP-9 median concentration at 1-year post transplantation tends to be higher in the CLAD group; however, the difference with the Stable recipients was not significant (236 vs 160 ng/ml) but at 2-year post transplantation MMP-9 was significantly higher in the CLAD group as compared to Stable (265 vs 132 ng/ml, p=0.01) (FIG. 5A).

TABLE 3
MMP-9 blood levels in CLAD vs Stable recipients
according to 3-year phenotype

| | pre-Tx | | 1-year | | 2-year | |
|---|---|---|---|---|---|---|
| | CLAD | Stable | CLAD | Stable | CLAD | Stable |
| minimum | 15 | 15 | 20 | 32 | 23 | 11 |
| 1st quartile | 184 | 171 | 81 | 74 | 106 | 68 |
| lower CI 95% | 298 | 330 | 150 | 125 | 187 | 102 |
| limit | | | | | | |
| median | 379 | 379 | 236 | 160 | 265 | 132 |
| upper CI 95% | 460 | 427 | 312 | 196 | 342 | 162 |
| limit | | | | | | |
| 3rd quartile | 790 | 813 | 556 | 303 | 404 | 250 |
| maximum | 2359 | 5059 | 1647 | 3296 | 1396 | 2769 |

### Higher MMP-9 Levels at 2-Year Post Transplantation for 5-Year CLAD Recipients

Similarly, a comparative analysis of MMP-9 plasmatic levels before transplantation, at 1-year and 2-year post transplantation comparing recipients who developed CLAD within 5 years and those who did not, was performed. Before transplantation, there was no difference between the two groups with comparable MMP-9 blood rate (369 vs 381 ng/ml). At 1-year post transplantation MMP-9 levels tend to be increased in the CLAD group, but the difference seen was not statistically significant (236 vs 152 ng/ml). However, at 2-year post transplantation, MMP-9 median blood level was higher in the CLAD group as compared to the Stable group (217 vs 131 ng/ml; p=0.005) (Table 4) (FIG. 5B).

TABLE 4
MMP-9 blood levels in CLAD vs Stable recipients
according to 5-year phenotype

| | pre-Tx | | 1-year | | 2-year | |
|---|---|---|---|---|---|---|
| | CLAD | Stable | CLAD | Stable | CLAD | Stable |
| minimum | 15 | 15 | 20 | 32 | 23 | 11 |
| 1st quartile | 180 | 172 | 72 | 77 | 88 | 71 |
| lower CI 95% limit | 306 | 330 | 181 | 118 | 170 | 104 |
| median | 369 | 381 | 236 | 152 | 217 | 131 |
| upper CI 95% limit | 433 | 433 | 291 | 186 | 264 | 158 |
| 3rd quartile | 723 | 824 | 476 | 257 | 391 | 201 |
| maximum | 3535 | 5059 | 3204 | 3296 | 2769 | 772 |

### MMP-9 Concentrations are Increased in the Year Before CLAD Onset

Secondly, a different analysis comparing recipient's MMP-9 blood levels taken in the year before CLAD with those taken at least one year before CLAD diagnosis was performed. When considering the 3-year phenotype, MMP-9 median plasma rate was superior when performed in the year before CLAD (256 vs 211 ng/ml; p=). When performing the same analysis according to the 5-year phenotype the difference was even more important and significant (285 vs 163 ng/ml; p=0.009). Thus, MMP-9 tends to increase as CLAD diagnostic gets closet (Table 5).

TABLE 5
MMP-9 blood levels performed the year before CLAD
or at least on year before CLAD

| | 3-year phenotype | | | 5-year phenotype | | |
|---|---|---|---|---|---|---|
| | <1 year | >1 year | | <1 year | >1 year | |
| | before | before | | before | before | |
| | CLAD | CLAD | Stable | CLAD | CLAD | Stable |
| minimum | 21 | 20 | 11 | 21 | 20 | 11 |
| 1st quartile | 105 | 77 | 71 | 104 | 61 | 73 |
| lower CI 95% | 195 | 104 | 122 | 230 | 116 | 113 |
| limit | | | | | | |
| median | 256 | 211 | 145 | 285 | 163 | 137 |
| upper CI 95% | 317 | 318 | 169 | 340 | 211 | 161 |
| limit | | | | | | |
| 3rd quartile | 446 | 565 | 273 | 496 | 381 | 244 |
| maximum | 1647 | 1503 | 3296 | 3204 | 2769 | 3296 |

### Predictive Performance of Blood MMP-9 for CLAD

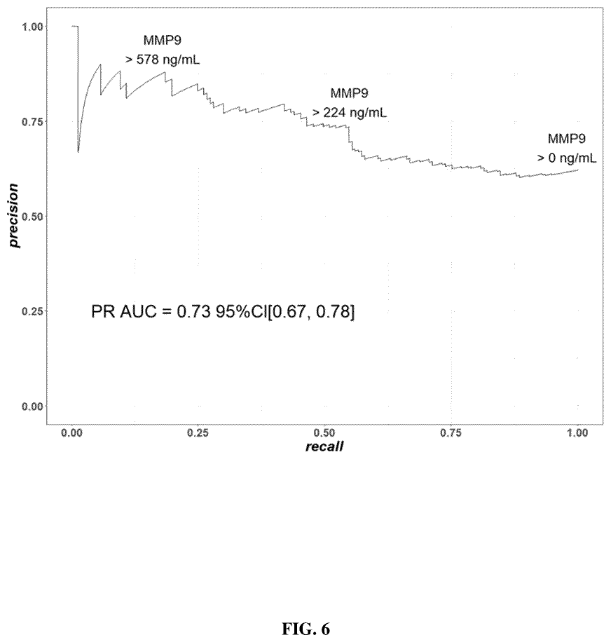

Finally, to determine the efficiency of MMP-9 plasma level at those time point to predict CLAD, a precision-recall analysis was performed in which plasma MMP-9 discriminated well between CLAD and Stable (precision AUC=0,73 95% CI [0,67-0,78] but has low sensibility. Global performance of the prediction is presented in FIG. 6. For example, a rate of 225 ng/ml had a precision of 75% and a recall of 50%, in other word, this threshold was correct 3 out of 4 times to identify a recipient who later developed CLAD but only engulfed half of the recipients who eventually developed CLAD.

### CONCLUSION

MMP-9 expression level measured two years after lung transplantation are predictive of CLAD.

1. A method for preventing Chronic Lung Allograft Dysfunction (CLAD) in a subject, comprising:
1) determining a subject as being at risk of having CLAD by:
a. obtaining at least one variable from the subject by measuring the level, amount or concentration of at least one biomarker in a biological sample from the subject, wherein said at least one biomarker is selected from the group comprising TCL1A, BLK, POU2AF1 and matrix metalloproteinase-9 (MMP-9),
b. obtaining at least one clinical data variable from the subject selected from the group comprising allograft rejection experience before sampling, maintenance treatment at sampling, induction treatment at transplantation, and mathematical combinations thereof,
c. obtaining a risk score by mathematically combining in a multivariate model:
i. said at least one variable obtained in step (a) and/or a reduction dimension of the level, amount or concentration mathematically obtained from at least two biomarkers measured in step (a); and
ii. said at least one clinical data variable obtained in step (b), and
d. determining the risk of Chronic Lung Allograft Dysfunction in the subject by comparing the obtained risk score with a reference risk score; and
2) implementing an adapted patient care depending on the risk of CLAD.

2. The method for preventing CLAD according to claim 1, comprising obtaining a risk score by mathematically combining in a multivariate model in step (c) at least the following variables:
a. the level, amount or concentration of TCL1A, BLK, and POU2AF1, and the clinical data variable allograft rejection experience before sampling, or
b. the level, amount or concentration of TCL1A, BLK, and POU2AF1, and the clinical data variable maintenance treatment at sampling, or
c. the level, amount or concentration of TCL1A, BLK, and POU2AF1, and the clinical data variable induction treatment at transplantation.

3. The method for preventing CLAD according to claim 1, wherein the subject is a lung transplanted subject.

4. The method for preventing CLAD according to claim 1, wherein-further comprising determining in step 1) a subject as being at risk of having a bronchiolitis obliterans syndrome (BOS).

5. The method for preventing CLAD according to claim 1, wherein-further comprising determining in step 1) a subject as being at risk of having a restrictive allograft syndrome (RAS).

6. The method for preventing CLAD according to claim 1, wherein the expression level of the at least one biomarker is measured in a biological sample from 18 months post-transplantation.

7. The method for preventing CLAD according to claim 1, wherein the biological sample is a blood sample.

8. The method for preventing CLAD according to claim 1, wherein said multivariate model is obtained from a Cox model, and/or wherein said multivariate model is time-fixed or time-dependent.

9. The method for preventing CLAD according to claim 1, wherein the method is computer implemented.

10. (canceled)

11. (canceled)

12. (canceled)

13. (canceled)

14. (canceled)

15. The method for preventing CLAD according to claim 1, wherein the expression level of the at least one biomarker is measured in a biological sample from 24 months post-transplantation.

16. The method for preventing CLAD according to claim 1, wherein said multivariate model is obtained from a Cox model, and/or wherein said multivariate model is time-dependent.

17. The method for preventing CLAD according to claim 1, wherein step 2) comprises: implementing an adapted patient care depending on the risk of CLAD comprising: administering at least one immunosuppressive and immunomodulatory therapies selected from the group comprising immunosuppressive and immunomodulatory drugs, anti-fibrotic treatment, cellular-based therapies, such as the use of mesenchymal stromal cells, endothelial progenitor cells, regulatory T cells (Tregs) or chimeric antigen receptors (CAR) Tregs, prophylactic treatment with azithromycin, total lymphoid irradiation (TLI) or extracorporeal photopheresis (ECP), current ant novel biotherapy such as anti-TNFα, anti-IL6R or JAKs inhibitors.

18. The method for preventing CLAD according to claim 1, wherein step 1) comprises a computer system determining a subject as being at risk of having CLAD.

19. The method for preventing CLAD according to claim 18, wherein the computer system comprises:
i) at least one processor, and
ii) at least one computer readable storage medium that at least one code readable by the processor, when executed but the processor, causes the processor to:
a. receive (i) input levels, amounts or concentrations of at least one biomarker, wherein said at least one biomarker is selected from the group comprising or consisting of TCL1A, BLK, POU2AF1 and matrix metalloproteinase-9 (MMP-9), and (ii) input values of at least one clinical data variable selected from a group comprising allograft rejection experience, maintenance treatment at sampling, and induction treatment at transplantation, and mathematical combinations thereof,
b. analyze and transform the input levels, amounts or concentrations and the input values, to derive a risk score,
c. generate an output, wherein the output is the risk score, and
d. provide the risk of Chronic Lung Allograft Dysfunction for the subject by comparing the obtained risk score with a reference risk score.
